(19) United States
(12) Patent Application Publication
(10) Pub. No.: US 2004/0106590 A1
(43) Pub. Date: Jun. 3, 2004

(54) Methods and reagents for treating infections of clostridium difficile and diseases associated therewith

(76) Inventor: Barry Eisenstein, Chestnut Hill, MA (US)

Correspondence Address: CLARK & ELBING LLP, 101 FEDERAL STREET, BOSTON, MA, 02110, US

(21) Appl. No.: 10/652,799

(22) Filed: Aug. 29, 2003

Related U.S. Application Data
(60) Provisional application No. 60/444,570, filed on Feb. 3, 2003.
(60) Provisional application No. 60/406,873, filed on Aug. 29, 2002.

(51) Int. Cl. A61K 31/33; A01N 25/00

(52) U.S. Cl. 514/183; 424/405

(57) ABSTRACT

The invention features methods and compositions including rifalazil for treating Clostridium difficile.

### CROSS REFERENCES TO RELATED APPLICATIONS

This application claims the benefit of U.S. Provisional Application Nos. 60/444,570, filed on Feb. 3, 2003, and 60/406,873, filed on Aug. 29, 2002, herein incorporated by reference.

### BACKGROUND OF THE INVENTION

The invention relates to the field of bacterial infections.

Clostridium (C.) difficile, a Gram-positive anaerobic bacterium, is recognized as the major causative agent of colitis (inflammation of the colon) and antibiotic-associated diarrhea that may occur following antibiotic intake. C. difficile infection represents one of the most common hospital (nosocomial) infections around the world. In the United States alone, it causes approximately three million cases of diarrhea and colitis per year. This bacterium is primarily acquired in hospitals and chronic care facilities following antibiotic therapy, and is the most frequent cause of outbreaks of diarrhea in hospitalized patients. One of the main characteristics of C. difficile-associated colitis is severe inflammation in the colonic mucosa associated with destruction of colonocytes.

The disease initially involves alterations of the beneficial bacteria, which are normally found in the colon, by antibiotic therapy. The alterations lead to colonization by C. difficile when this bacterium or its spores are present in the environment. In hospitals or nursing home facilities where C. difficile is prevalent and patients frequently receive antibiotics, C. difficile infection is very common. In contrast, individuals treated with antibiotics as outpatients have a much smaller risk of developing C. difficile infection.

Laboratory studies show that when C. difficile colonize the intestinal tract, they release two potent toxins, toxin A and toxin B, which bind to certain receptors in the lining of the colon and ultimately cause diarrhea and inflammation of the large intestine, or colon (colitis). Thus, the toxins are involved in the pathogenesis, or development of the disease.

Although C. difficile infection usually responds well to treatment with metronidazole or vancomycin, approximately 15 to 20% of patients will experience re-appearance of diarrhea and other symptoms weeks or even months after initial therapy has been discontinued. The usual therapy for relapse is to repeat the 10 to 14 day course of either metronidazole or vancomycin. A subset of patients continues to relapse whenever antibiotics are discontinued and this represents a therapeutic challenge.

Thus, there is a need for improved methods for treating C. difficile infections.

### SUMMARY OF THE INVENTION

The invention features a method for treating a subject having antibiotic-associated bacterial diarrhea or an infection of Clostridium (C.) difficile, or preventing the disease or infection in the subject. The method includes the step of administering to the subject an effective amount of rifalazil to treat the subject. The dosage of rifalazil can range from 0.01 mg to 1000 mg. The dosage of rifalazil is normally about 1 to 1000 mg (desirably about 1 to 100 mg, more desirably about 1 to 50 mg, and even more desirably about 1 to 25 mg). The rifalazil may be given daily (e.g., once, twice, three times, or four times daily) or less frequently (e.g., once every other day, once or twice weekly, or monthly). Rifalazil is administered for a length of time sufficient to treat the subject. Treatment may be for 1 to 31 days, desirably 1 to 21 days, 1 to 14 days or even 1, 3, 5, or 7 days. If desired, treatment can continue for up to a year or even for the lifetime of the subject. In one example, rifalazil is administered at an initial dose of between 5 and 100 mg, followed by subsequent doses of between 1 and 50 mg for 3 to 7 days. A single dose of rifalazil (e.g., in a dosage of between 1 and 100 mg) can also be employed in the method of the invention. The rifalazil may be administered orally, intravenously, subcutaneously, or rectally.

The method of the invention may be employed as an initial treatment of a subject having or being at risk for developing antibiotic-associated bacterial diarrhea or an infection of C. difficile, or it may be employed to treat subjects for whom the initial treatment (e.g., with metronidazole, vancomycin, rifampicin, rifabutin, rifapentine, and rifaximin) has failed to fully treat the antibiotic-associated bacterial diarrhea or an infection of C. difficile. The method may be employed, for example, when the subject is colonized with C. difficile organisms that are resistant to one or more of metronidazole, vancomycin, rifampicin, rifabutin, rifapentine, and rifaximin.

If desired, rifalazil can be administered with one or more additional antibiotics or with an agent that binds toxin A or toxin B (e.g., the non-absorbed toxin binding polymer GT160-246; U.S. Pat. No. 6,270,755). Preferred examples of additional antibiotics are metronidazole, gentamicin, daptomycin, azithromycin, quinupristin, dalfopristin, linezolid, teicoplanin, ciprofloxacin, and vancomycin.

In certain embodiments of the invention, the method includes administering rifalazil and vancomycin simultaneously or sequentially. Rifalazil and vancomycin can be administered within fourteen days of each other, or within five days, three days, or within twenty-four hours of each other. If desired, either rifalazil or vancomycin, or both can be administered orally. Preferred dosages for vancomycin can range from 20 to 2000 mg per day, preferably from 125 to 2000 mg per day, most preferably from 500 to 2000 mg per day.

The invention also features a method of treating a subject having antibiotic-associated bacterial diarrhea or an infection of Clostridium (C.) difficile, or preventing the disease or infection in the subject. The method includes the step of administering to the subject a composition comprising rifalazil and vancomycin, which can be suitable for oral or intravenous administration. The unit dosages for rifalazil can range from 0.01 to 100 mg (e.g., between 1 and 100 mg, or between 1 and 50 mg, or between 1 and 25 mg, or between 1 and 5 mg), and the unit dosages for vancomycin can range from 125 to 2000 mg, preferably from 500 to 2000 mg.

The invention also features a pharmaceutical pack comprising (i) rifalazil in an amount effective to treat a subject having antibiotic-associated bacterial diarrhea or an infection of C. difficile; and (ii) instructions for administering the rifalazil to a subject for treating or preventing a C. difficile infection. Desirably, the rifalazil is in unit amounts of between 0.01 and 1000 mg (e.g., between 1 and 100 mg, or between 1 and 50 mg, or between 1 and 25 mg, or between 1 and 5 mg), and is present in amounts sufficient to treat for at least 1, 3, 5, 7, 10, 14, 21, or 31 days.

The pharmaceutical pack of the invention can further comprise one or more antibiotics. Preferred examples of the additional antibiotic include metronidazole, gentamicin, daptomycin, azithromycin, quinupristin, dalfopristin, linezolid, teicoplanin, ciprofloxacin., and vancomycin. Typical dosages for vancomycin range from 20 to 2000 mg, preferably from 125 to 2000 mg.

Exemplary additional antibiotics that can be administered in the methods of the invention or included in the pharmaceutical pack of the invention are β-lactams such as penicillins (e.g., penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin), cephalosporins (e.g., cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, and BAL9141), carbapenams (e.g., imipenem, ertapenem, and meropenem), and monobactams (e.g., astreonam); β-lactamase inhibitors (e.g., clavulanate, sulbactam, and tazobactam); aminoglycosides (e.g., streptomycin, neomycin, kanamycin, paromycin, gentamicin, tobramycin, amikacin, netilmicin, spectinomycin, sisomicin, dibekalin, and isepamicin); tetracyclines (e.g., tetracycline, chlortetracycline, demeclocycline, minocycline, oxytetracycline, methacycline, and doxycycline); lipopetides (e.g., daptomycin); macrolides (e.g., erythromycin, azithromycin, and clarithromycin); ketolides (e.g., telithromycin, ABT-773); lincosamides (e.g., lincomycin and clindamycin); glycopeptides (e.g., vancomycin, oritavancin, dalbavancin, and teicoplanin); streptogramins (e.g., quinupristin and dalfopristin); sulphonamides (e.g., sulphanilamide, para-aminobenzoic acid, sulfadiazine, sulfisoxazole, sulfamethoxazole, and sulfathalidine); oxazolidinones (e.g., linezolid); quinolones (e.g., nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin); rifamycins (e.g., rifampicin, rifabutin, rifapentine, and rifaximin); metronidazole; garenoxacin; ramoplanin; faropenem; polymyxin; tigecycline, AZD2563; and trimethoprim.

By “an effective amount” is meant the amount of rifalazil or rifalazil in combination with one or more additional antibiotics required to result in the C. difficile being eradicated from the subject, or to prevent an infection of C. difficile, as determined by a diagnostic test that detects C. difficile. One example of a diagnostic test is the use of a commercially available enzyme-linked immunoassay (ELISA; Immunocard; Meridian Diagnostics, Inc., Cincinnati, Ohio) to detect the presence of C. difficile toxin A protein in cecal content extracts. Another example of a diagnostic test is the use of a cytotoxicity assay using human fibroblast cells (Toxi-Titer; Bartels, Inc., Issaquah, Wash.) to detect the presence of C. difficile toxin B. Both of these examples can be found in McVay and Rolfe (Antimicrobial Agents and Chemo. 44:2254-2258, 2000). An “effective amount” can also mean the amount of rifalazil or rifalazil in combination with one or more additional antibiotics required to reduce the symptoms of the C. difficile-associated disease in a subject or animal model. Symptoms include diarrhea, weight loss, lethargy, and ruffled fur in specific animal models. Standard assays present in the art can be used to measure the symptoms of disease (for examples of assays see Boon and Beale, Drugs Suppl. 5:57-63, 1985 and McVay and Rolfe, supra). An “effective amount” of rifalazil or rifalazil in combination with one or more additional antibiotics reduces the symptoms of C. difficile-associated disease in a subject by 20%, preferably, 30% or 40%, more preferably, 50% or 60%, and most preferably, 70%, 80%, 90%, or more, as compared to an untreated subject.

“Rifalazil” means 3′-hydroxy-5′-(4-isobutyl-1-piperazinyl) benzoxazinorifamycin, also known as KRM-1648 or ABI1648. Methods of making rifalazil and microgranulated formulations thereof are described in U.S. Pat. Nos. 4,983,602 and 5,547,683, respectively.

“Antibiotic-associated bacterial diarrhea” means a condition in which antibiotic therapy disturbs the balance of the microbial flora of the gut, allowing pathogenic organisms such as C. difficile to flourish. These organisms cause diarrhea. Antibiotic-associated bacterial diarrhea includes such conditions as C. difficile associated diarrhea (CDAD) and pseudomembranous colitis.

“Pseudomembranous colitis,” also known as pseudomembranous enterocolitis or enteritis, means the inflammation of the mucous membrane of both small and large intestine with the formation and passage of pseudomembranous material (composed of fibrin, mucous, necrotic epithelial cells and leukocytes) in the stools.

The term “lower gastrointestinal tract” means the lower part of the small intestine (ileum) and the colon.

The term “enteric coating” means a coating surrounding a core, the solubility of the coating being dependent on the pH in such a manner that it substantially prevents the release of a drug in the stomach, but permits the release of the drug at some stage after the formulation has emptied from the stomach. The term “pH-sensitive enteric polymer” means a polymer the solubility of which is dependent on the pH so that it is insoluble in the gastric juice but dissolves at some stage after the formulation has emptied from the stomach.

By “subject” is meant any warm-blooded animal including but not limited to a human, cow, horse, pig, sheep, goat, bird, mouse, rat, dog, cat, monkey, baboon, or the like. It is most preferred that the subject be a human.

### DETAILED DESCRIPTION OF THE INVENTION

We have discovered that administration of rifalazil alone or in combination with one or more additional antibiotics is effective to treat a subject having antibiotic-associated bacterial diarrhea or an infection of Clostridium (C.) difficile.

The dosage of rifalazil can range from about 0.01 to 1000 mg. The dosage of rifalazil is normally about 1 to 1000 mg (desirably about 1 to 100 mg, more desirably about 1 to 50 mg, and even more desirably about 1 to 25 mg). The rifalazil may be given daily (e.g., once, twice, three times, or four times daily) or less frequently (e.g., once every other day, once or twice weekly, or monthly). The administration of rifalazil may be by any suitable means that results in an effective amount of the compound reaching the target region. The compound may be contained in any appropriate amount in any suitable carrier substance, and is generally present in an amount of 1-95% by weight of the total weight of the composition. In one embodiment, the composition is provided in a dosage form that is suitable for oral administration, e.g., a tablet, capsule, pill, powder, granulate, suspension, emulsion, solution, or gel. Alternatively, the composition may be formulated for intravenous or rectal administration. An intravenous formulation of rifalazil is described in Utility U.S. application Ser. No. 10/453,155 (filed Jun. 3, 2003). The pharmaceutical composition can generally be formulated according to conventional pharmaceutical practice (see, e.g., Remington: The Science and Practice of Pharmacy (20th ed.), ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, N.Y.).

Pharmaceutical compositions according to the invention may be formulated to release rifalazil substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain drug action during a predetermined time period by maintaining a relatively, constant, effective drug level in the body with concomitant minimization of undesirable side effects associated with fluctuations in the plasma level of the active drug substance (sawtooth kinetic pattern); (iv) formulations that localize drug action by, e.g., spatial placement of a controlled release composition adjacent to or in the diseased tissue or organ; and (v) formulations that target drug action by using carriers or chemical derivatives to deliver the drug to a particular target cell type. Controlled release formulations that may be employed in the method of the invention include those described in U.S. Pat. Nos. 5,007,790, 5,525,634, 5,582,837, 5,811,388, 5,849,327, 5,962,024, 5,968,554, 5,972,389, 6,319,518, 6,340,475, 6,488,962, and 6,506,407, each of which is hereby incorporated by reference.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug substance in a predetermined pattern (e.g., in order to achieve a controlled release formulation) or it may be adapted not to release the active drug substance until after passage of the stomach; this may be achieved by means of an enteric coating (e.g., a pH-sensitive enteric polymer). Desirably, a substantial amount of the drug is released in the lower gastrointestinal tract. The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or a coating based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

The solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, supra.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may be constructed to release the active drug by controlling the dissolution and/or the diffusion of the active drug substance.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, camauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl inethacrylate, polyvinyl chloride, polyethylene, and/or halogenated fluorocarbon.

A controlled release composition containing rifalazil may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Liquids for Oral Administration

Powders, dispersible powders, or granules suitable for preparation of an aqueous suspension by addition of water are convenient dosage forms for oral administration of rifalazil. Formulation as a suspension provides the active ingredient in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

Formulations and Dosages for Combination Therapies

Rifalazil can be administered to a subject having antibiotic associated bacterial diarrhea or an infection of C. difficile in conjunction with one or more additional antibiotics. Rifalazil can be administered before, during, or after administration of the additional antibiotics, or any combination thereof. If desired, the administration of rifalazil can be continued while the additional antibiotic is being administered.

Exemplary antibiotics that can be administered in the methods of the invention are β-lactams such as penicillins (e.g., penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin), cephalosporins (e.g., cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, and BAL9141), carbapenams (e.g., imipenem, ertapenem, and meropenem), and monobactams (e.g., astreonam); β-lactamase inhibitors (e.g., clavulanate, sulbactam, and tazobactam); aminoglycosides (e.g., streptomycin, neomycin, kanamycin, paromycin, gentamicin, tobramycin, amikacin, netilmicin, spectinomycin, sisomicin, dibekalin, and isepamicin); tetracyclines (e.g., tetracycline, chlortetracycline, demeclocycline, minocycline, oxytetracycline, methacycline, and doxycycline); lipopetides (e.g., daptomycin); macrolides (e.g., erythromycin, azithromycin, and clarithromycin); ketolides (e.g., telithromycin, ABT-773); lincosamides (e.g., lincomycin and clindamycin); glycopeptides (e.g., vancomycin, oritavancin, dalbavancin, and teicoplanin); streptogramins (e.g., quinupristin and dalfopristin); sulphonamides (e.g., sulphanilamide, para-aminobenzoic acid, sulfadiazine, sulfisoxazole, sulfamethoxazole, and sulfathalidine); oxazolidinones (e.g., linezolid); quinolones (e.g., nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin); rifamycins (e.g., rifampicin, rifabutin, rifapentine, and rifaximin); metronidazole; garenoxacin; ramoplanin; faropenem; polymyxin; tigecycline, AZD2563; and trimethoprim.

These antibiotics can be used in the dose ranges and formulations currently known and used for these agents. Different concentrations may be employed depending on the clinical condition of the subject, the goal of therapy (treatment or prophylaxis), the anticipated duration, and the severity of the C. difficile infection. Additional considerations in dose selection include the type of infection, age of the subject (e.g., pediatric, adult, or geriatric), general health, and comorbidity. Determining what concentrations to employ are within the skills of the pharmacist, medicinal chemist, or medical practitioner. Typical dosages and frequencies are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002).

In one example rifalazil is administered in combination with vancomycin. Either the rifalazil or the vancomycin or both may be given daily (e.g., once, twice, three times, or four times daily) or less frequently (e.g., once every other day, once every three days, once or twice weekly, or monthly). Typical daily dosages for vancomycin range from 20 mg to 2 gm, preferably 125 mg to 2 gm, or 500 mg to 2 gm, but it may be administered in any higher tolerated amounts as necessary. Daily dosages of vancomycin can be can be distributed over one to four doses. Exemplary daily oral dosages include from 500 mg to 2 gm distributed over one to four doses for adult subjects and 40 mg/kg distributed over one to four doses for pediatric subjects. Intravenous administration can be given as a one-time bolus per 24-hour period, or for any subset of time over the 24-hour period (e.g., half an hour, one hour, two hours, four hours, up to 24 hours).

For combination therapy, the rifalazil and the additional antibiotic can be administered simultaneously or sequentially. For sequential administration, the rifalazil can be administered before, during, or after administration of the additional antibiotic, or any combination thereof. In one example, vancomycin is administered for five days and rifalazil is administered as a single dose on the sixth day. In another example, vancomycin and rifalazil are administered simultaneously on day one followed by administration of vancomycin for an additional six days. These examples are provided to illustrate two potential combinations for sequential therapy. They are not intended to limit the invention in any way.

For combination therapy, the dosage and the frequency of administration of each component of the combination can be controlled independently. For example, one of the compounds (i.e., rifalazil or the additional antibiotic) may be administered three times per day, while the second compound may be administered once per day. The compounds may also be formulated together such that one administration delivers both compounds. In one particularly preferred embodiment, rifalazil is administered orally or intravenously, while the additional antibiotic is administered orally. In another preferred embodiment, both rifalazil and the additional antibiotic (e.g., vancomycin) are administered orally.

The following examples are shown to illustrate, but not to limit the present invention.

### EXAMPLES

### Example 1

### Animal Models of C. Difficile-Associated Disease

Optimal dosages and formulations of rifalazil alone, or in combination with a second drug compound, can be determined using standard animal models known in the art. One example of an animal model for C. difficile associated disease is the Golden Syrian hamster. To determine the optimal dosage regimen of rifalazil, Golden Syrian hamsters are injected subcutaneously with clindamycin phosphate (10 mg/kg) followed, 24 hours later, by oral gavage with 10⁵ colony forming units (CFU) of C. difficile. Antibiotic treatment is then administered orally, intravenously, or intraperitoneally, either simultaneously or 24 hours after C. difficile administration. Animals are monitored for survival, weight variations, identification of C. difficile toxins in cecal content, and histologic damage to ceca as compared to animals treated with a prophylactic protocol using standard methods known in the art (see, for example, Anton P. M. et al., Abstract ID No. 102471, Publishing ID No. T1741, presented at the American Gastroenterological Association Meeting, May 17-22, 2003; Anton P. M. et al., Gastroenterology 124:A558, 2003).

### Example 2

### Minimum Inhibitory Concentration of Rifalazil (ABI1648) for 31 Strains of C. Difficile

The minimum inhibitory concentration (MIC) of rifalazil was determined for 31 strains of C. difficile. All C. difficile isolates were collected in U.S. hospitals from 1987 to 1992. MICs were determined by agar dilution using NCCLS defined methodology (National Committee for Clinical Laboratory Standards, Methods for Antimicrobial Susceptibility Testing of Anaerobic Bacteria, Approved Standard M11-A5, Fifth Ed., 2001). Rifalazil and metronidazole were tested over an eleven doubling dilution concentration range.

Agar dilution testing panels were prepared on the day of testing. Agar containing vitamin K and hemin was melted and cooled to 50° C. and 1 mL of blood supplement was added at this time. Prepared concentrations of each antimicrobial agent were added to the appropriate agar tube and mixed thoroughly. The agar mixture was poured into round petri dishes and allowed to solidify; plates were then placed in an incubator at 35-37° C. for 30 minutes to allow for excess moisture on the agar surface to evaporate.

For agar dilution susceptibility testing, isolates frozen at −70° C. were thawed and subcultured twice onto Brucella agar, with incubation at 35° C. in 4-7% CO₂ for 48-72 hours following each subculture. A bacterial suspension was prepared in Brucella broth and adjusted to the density of a 0.5 McFarland standard. Agar plates with appropriate concentrations of antimicrobial agents were inoculated with 1-2 μl of bacterial suspension (10⁵ CFU per spot). Upon inoculation, plates were inverted and incubated at 35-37° C. in an anaerobic environment. Plates were read after 48 hours of incubation and MICs were determined.

MICs for rifalazil were compared to MICs for metronidazole which were also determined for the 31 strains of C. difficile. These results are shown in Table 1.

TABLE 1
MICs (μg/mL) for rifalazil and metronidazole on 31 strains of C. difficile.

| Sample | | Metron- | | |
|---|---|---|---|---|
| # | Organism ID | idazole | ABI1648 | Identity |
| QC | B. frag 25285 | 1 | 0.03* | NA |
| QC | B. theta 29741 | 2 | 0.03 | NA |
| 1 | C. difficile 18 | 0.5 | 0.001 | Different |
| 2 | C. difficile 22 | 0.5 | 0.001 | Different |
| 3 | C. difficile 28 | 0.5 | 0.002 | Different |
| 4 | C. difficile 40 | 0.5 | 0.001 | Different |
| 5 | C. difficile 44 | 0.5 | 0.001 | Different |
| 6 | C. difficile 50 | 0.5 | 0.002 | Different |
| 7 | C. difficile 60 | 0.5 | 0.002 | Not typable |
| 8 | C. difficile 70 | 0.25 | 0.001 | Not typable |
| 9 | C. difficile 80 | 0.5 | >20.5 | Different |
| 10 | C. difficile 101 | 0.5 | 0.004 | Different |
| 11 | C. difficile 107 | 0.5 | 0.001 | Different |
| 12 | C. difficile 113 | 0.5 | 0.004 | Different |
| 13 | C. difficile 116 | 0.25 | 0.002 | Not typable |
| 14 | C. difficile 143 | 0.5 | <=0.0005 | Different |
| 15 | C. difficile 146 | 0.5 | 0.001 | Different |
| 16 | C. difficile 154 | 0.25 | <=0.0005 | Not typable |
| 17 | C. difficile 166 | 0.5 | 0.001 | Different |
| 18 | C. difficile 199 | 0.25 | <=0.0005 | Different |
| 19 | C. difficile 65C | 0.5 | 0.002 | Not typable |
| 20 | C. difficile 5-5-03 | 0.5 | 0.002 | Not typable |
| 21 | C. difficile 6-5-03 | 0.5 | 0.002 | Different |
| 22 | C. difficile 7-5-03 | 0.25 | 0.002 | Different |
| 23 | C. difficile 11-5-03 | 0.5 | 0.001 | Not typable |
| 24 | C. difficile 13-5-03 | 0.5 | <=0.0005 | Different |
| 25 | C. difficile 14-5-03 | 0.5 | 0.001 | Not typable |
| 26 | C. difficile 16-5-03 | 0.5 | 0.008 | Different |
| 27 | C. difficile 17-5-03 | 0.25 | 0.002 | Different |
| 28 | C. difficile 19-5-03 | 0.5 | 0.002 | Different |
| 29 | C. difficile 22-5-03 | 0.25 | 0.001 | Different |
| 30 | C. difficile 29-5-03 | 0.5 | >0.5 | Different |
| 31 | QC C. diff ATCC 43255 | 0.5 | 0.002 | Different |

*It should be noted that Fujii et al., (Antimicrob. Agents Chemother. 38:1118-1122, 1994) obtained a value of 1.0 for this organism, a value which differs from that described in Table 1.

These data demonstrate that rifalazil was much more effective than metronidazole against almost all 31 strains of C. difficile. MICs for rifalazil were in the range of 0.0005 to 0.5 μg/mL while MICs for metronidazole were in the range of 0.25 to 0.5 μg/mL. Furthermore, identity analyses of these strains were performed, where possible, and indicate that the strains of C. difficile tested were not identical.

Strains 80 and 29-5-30 appeared to be less susceptible to metronidazole and rifalazil and therefore indicate the need for combination treatments described herein for the effective treatment of some strains of C. difficile.

### Other Embodiments

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in microbiology or related fields are intended to be within the scope of the invention.

What is claimed is:

1. A method for treating a subject having an infection of Clostridium difficile or preventing an infection of Clostridium difficile in said subject, said method comprising administering to said subject an effective amount of rifalazil.

2. The method of claim 1, wherein said rifalazil is administered in an amount between 0.01 and 1000 mg/day.

3. The method of claim 2, wherein said rifalazil is administered in an amount between 1 and 100 mg/day.

4. The method of claim 3, wherein said rifalazil is administered in an amount between 1 and 50 mg/day.

5. The method of claim 4, wherein said rifalazil is administered in an amount between 5 and 25 mg/day.

6. The method of claim 1, wherein said rifalazil is administered for one to fourteen days.

7. The method of claim 6, wherein said rifalazil is administered for three to seven days.

8. The method of claim 1, wherein said rifalazil is administered as a single dose.

9. The method of claim 1, wherein said rifalazil is administered at an initial dose of between 5 and 100 mg, followed by subsequent doses of between 1 and 50 mg for three to seven days.

10. The method of claim 1, wherein said infection of Clostridium difficile comprises a strain of Clostridium difficile that is resistant to one or more antibiotics selected from the group consisting of vancomycin, rifampicin, rifabutin, rifapentine, rifaximin, and metronidazole.

11. The method of claim 1, wherein said rifalazil is administered orally, intravenously, subcutaneously, or rectally.

12. The method of claim 1, further comprising administering to said subject an agent that binds Clostridium difficile toxin A or toxin B.

13. The method of claim 1, further comprising administering to said subject one or more antibiotics selected from the group consisting of β-lactams, β-lactamase inhibitors, aminoglycosides, tetracyclines, lipopetides, macrolides, ketolides, lincosamides, streptogramins, sulphonamides, oxazolidinones, quinolones, rifamycins, glycopeptides, metronidazole, garenoxacin, ramoplanin, faropenem, polymyxin, tigecycline, AZD2563, and trimethoprim.

14. The method of claim 13, wherein said β-lactam is selected from the group consisting of penicillins, cephalosporins, carbapenams, and monobactams.

15. The method of claim 14, wherein said penicillin is selected from the group consisting of penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin.

16. The method of claim 14, wherein said cephalosporin is selected from the group consisting of cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, and BAL9141.

17. The method of claim 14, wherein said carbapenam is selected from the group consisting of imipenem, ertapenem, and meropenem.

18. The method of claim 14, wherein said monobactam is astreonam.

19. The method of claim 13, wherein said β-lactamase inhibitor is selected from the group consisting of clavulanate, sulbactam, and tazobactam.

20. The method of claim 13, wherein said aminoglycoside is selected from the group consisting of streptomycin, neomycin, kanamycin, paromycin, gentamicin, tobramycin, amikacin, netilmicin, spectinomycin, sisomicin, dibekalin, and isepamicin.

21. The method of claim 20, wherein said aminoglycoside is gentamicin.

22. The method of claim 13, wherein said tetracycline is selected from the group consisting of tetracycline, chlortetracycline, demeclocycline, minocycline, oxytetracycline, methacycline, and doxycycline.

23. The method of claim 13, wherein said lipopetide is daptomycin.

24. The method of claim 13, wherein said macrolide is selected from the group consisting of erythromycin, azithromycin, and clarithromycin.

25. The method of claim 24, wherein said macrolide is azithromycin.

26. The method of claim 13, wherein said ketolide is selected from the group consisting of telithromycin and ABT-773.

27. The method of claim 13, wherein said lincosamide is selected from the group consisting of lincomycin and clindamycin.

28. The method of claim 13, wherein said streptogramin is selected from the group consisting of quinupristin and dalfopristin.

29. The method of claim 13, wherein said sulphonamide is selected from the group consisting of sulphanilamide, para-aminobenzoic acid, sulfadiazine, sulfisoxazole, sulfamethoxazole, and sulfathalidine.

30. The method of claim 13, wherein said oxazolidinone is linezolid.

31. The method of claim 13, wherein said quinolone is selected from the group consisting of nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin.

32. The method of claim 31, wherein said quinolone is ciprofloxacin.

33. The method of claim 13, wherein said rifamycin is selected from the group consisting of rifampicin, rifabutin, rifapentine, and rifaximin.

34. The method of claim 13, wherein said antibiotic is metronidazole.

35. The method of claim 13, wherein said glycopeptide is selected from the group consisting of vancomycin, oritavancin, dalbavancin, and teicoplanin.

36. The method of claim 35, wherein said glycopeptide is teicoplanin.

37. The method of claim 35, wherein said glycopeptide is vancomycin.

38. The method of claim 37, wherein said rifalazil and vancomycin are administered simultaneously.

39. The method of claim 37, wherein said rifalazil and vancomycin are administered sequentially.

40. The method of claim 37, wherein said rifalazil and vancomycin are administered within fourteen days of each other.

41. The method of claim 40, wherein said rifalazil and vancomycin are administered within five days of each other.

42. The method of claim 41, wherein said rifalazil and vancomycin are administered within three days of each other.

43. The method of claim 42, wherein said rifalazil and vancomycin are administered within twenty-four hours of each other.

44. The method of claim 37, wherein at least one of said rifalazil and said vancomycin is administered orally.

45. The method of claim 37, wherein both of said rifalazil and said vancomycin are administered orally.

46. The method of claim 37, wherein said vancomycin is administered in an amount between 125 and 2000 mg per day.

47. The method of claim 46, wherein said vancomycin is administered in an amount between 500 and 2000 mg per day.

48. A method of treating a subject having an infection of Clostridium difficile or preventing an infection of Clostridium difficile in said subject, said method comprising administering to said subject a composition comprising rifalazil and vancomycin.

49. The method of claim 48, wherein said composition is suitable for oral administration.

50. The method of claim 48, wherein said composition is suitable for intravenous administration.

51. The method of claim 48, wherein said rifalazil is in a unit dosage amount between 0.01 and 100 mg, and said vancomycin is in a unit dosage amount between 125 and 2000 mg.

52. The method of claim 51, wherein said rifalazil is in a unit dosage amount between 1 and 50 mg, and said vancomycin is in a unit dosage amount between 500 and 2000 mg.

53. The method of claim 52, wherein said rifalazil is in a unit dosage amount between 1 and 25 mg, and said vancomycin is in a unit dosage amount between 500 and 2000 mg.

54. A pharmaceutical pack comprising (i) rifalazil in an amount effective to treat a subject having an infection of Clostridium difficile or prevent an infection of Clostridium difficile in said subject; and (ii) instructions for administering said rifalazil to said subject for treating or preventing a Clostridium difficile infection.

55. The pharmaceutical pack of claim 54, wherein said rifalazil is in a unit dosage amount between 0.01 and 100 mg.

56. The pharmaceutical pack of claim 55, wherein said rifalazil is in an amount between 1 and 50 mg.

57. The pharmaceutical pack of claim 56, wherein said rifalazil is in an amount between 1 and 25 mg.

58. The pharmaceutical pack of claim 57, wherein said rifalazil is in an amount between 5 and 25 mg.

59. The pharmaceutical pack of claim 54, further comprising one or more antibiotics selected from the group consisting of β-lactams, β-lactamase inhibitors, aminoglycosides, tetracyclines, lipopetides, macrolides, ketolides, lincosamides, streptogramins, sulphonamides, oxazolidinones, quinolones, rifamycins, glycopeptides, metronidazole, garenoxacin, ramoplanin, faropenem, polymyxin, tigecycline, AZD2563, and trimethoprim.

60. The pharmaceutical pack of claim 59, wherein said β-lactam is selected from the group consisting of penicillins, cephalosporins, carbapenams, and monobactams.

61. The pharmaceutical pack of claim 59, wherein said aminoglycoside is selected from the group consisting of streptomycin, neomycin, kanamycin, paromycin, gentamicin, tobramycin, amikacin, netilmicin, spectinomycin, sisomicin, dibekalin, and isepamicin.

62. The pharmaceutical pack of claim 61, wherein said aminoglycoside is gentamicin.

63. The pharmaceutical pack of claim 59, wherein said lipopetide is daptomycin.

64. The pharmaceutical pack of claim 59, wherein said macrolide is selected from the group consisting of erythromycin, azithromycin, and clarithromycin.

65. The pharmaceutical pack of claim 64, wherein said macrolide is azithromycin.

66. The pharmaceutical pack of claim 59, wherein said streptogramin is selected from the group consisting of quinupristin and dalfopristin.

67. The pharmaceutical pack of claim 59, wherein said oxazolidinone is linezolid.

68. The pharmaceutical pack of claim 59, wherein said quinolone is selected from the group consisting of nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin.

69. The pharmaceutical pack of claim 68, wherein said quinolone is ciprofloxacin.

70. The pharmaceutical pack of claim 59, wherein said rifamycin is selected from the group consisting of rifampicin, rifabutin, rifapentine, and rifaximin.

71. The pharmaceutical pack of claim 59, wherein said glycopeptide is selected from the group consisting of vancomycin, oritavancin, dalbavancin, and teicoplanin.

72. The pharmaceutical pack of claim 71, wherein said glycopeptide is teicoplanin.

73. The pharmaceutical pack of claim 71, wherein said glycopeptide is vancomycin.

74. The pharmaceutical pack of claim 73, wherein said vancomycin is in an amount between 125 and 2000 mg.

75. The pharmaceutical pack of claim 73, wherein said vancomycin is in an amount between 500 and 2000 mg.
